(19) United States
(12) Patent Application Publication
(10) Pub. No.: US 2012/0121516 A1
(43) Pub. Date: May 17, 2012

(54) Emulsion Activatable by Ultrasounds and Method for Producing Same

(75) Inventors: Patrick Tabeling, L'Hay les Roses (FR); Mickael Tanter, Bagneux (FR); Nicolas Pannacci, Paris (FR); Olivier Couture, Paris (FR); Mathias Fink, Meudon (FR)

(73) Assignee: Centre National de la Recherche Scientifique- CNRS, Paris (FR)

(21) Appl. No.: 13/382,745

(22) PCT Filed: Jul. 7, 2010
(86) PCT No.: PCT/FR10/51439

§ 371 Date: Jan. 6, 2012

(30) Foreign Application Priority Data
Jul. 17, 2009 (FR) 0955001

(51) Int. Cl. A61K 49/22; A61P 35/00; A61K 9/14; A61K 31/7088; B82Y 40/00; B82Y 5/00

(52) U.S. Cl. 424/9.5; 424/490; 514/44 R; 977/773; 977/906; 977/929; 977/900

(57) ABSTRACT

The invention relates to an emulsion that can be activated by ultrasounds, comprising, in an emulsion in an aqueous solution, microparticles having a diameter of less than 10 μm and containing an active agent and a gaseous precursor in a liquid form, encapsulated by a first emulsifier. The microparticles contain nanoparticles smaller than 1 μm, in an emulsion in the gaseous precursor, each nanoparticle comprising an inner liquid that contains the active agent and is encapsulated by a second emulsifier.

### FIELD OF THE INVENTION

The invention relates to emulsions that are activatable by ultrasound, as well as to the methods for producing them.

### BACKGROUND OF THE INVENTION

Emulsions activatable by ultrasound are currently known. These are used, for example, to transport a drug to a target area of the human body for local activation. Such known emulsions may, for example, be in the form of an aqueous solution containing microparticles of gas or gaseous precursors in suspension, encapsulated by a surfactant and containing the drug to be transported. During use, this solution is injected into a patient, then, after diffusion in the circulatory system, the microparticles are ruptured in the target area by focusing ultrasound on the target area. The drug contained in the microparticles is therefore released only in the target area, while the remainder of the microparticles are eliminated by the patient's metabolism.

An example of an emulsion of this type is given in patent US-A-2002/159952 (Unger).

A disadvantage of this type of emulsion, however, is that the active agent transported by the microparticles is at the surface of these microparticles, which limits the amount of active agent transported.

Patent WO2007/010442 describes microparticles encapsulated by a polymer membrane, containing a liquid gaseous precursor activatable by ultrasound and a hydrophobic active agent dissolved in an oil, forming a phase distinct from the gaseous precursor. This type of microparticle, however, seems to be very difficult or even impossible to achieve in practice, does not allow an optimum load of the active agent, and presents a risk of involuntary release of active agent. Lastly, this patent requires the use of a hydrophobic active agent, which greatly limits the applications for this technique.

### OBJECT AND SUMMARY OF THE INVENTION

A particular object of the invention is to overcome the above disadvantages.

For this purpose, the invention proposes an emulsion activatable by ultrasound, comprising, in emulsion in an aqueous solution, microparticles having a diameter of less than 20 μm comprising an active agent (marker or drug) and a gaseous precursor in a liquid form activatable by ultrasound, encapsulated by a first emulsifier,

wherein the microparticles contain nanoparticles having a diameter of less than 5 μm in emulsion in the gaseous precursor, each nanoparticle comprising an inner liquid which contains the active agent and which is encapsulated by a second emulsifier, said gaseous precursor forming a barrier against the diffusion of the active agent.

The active agent is therefore transported in the form of a double emulsion and no longer in the form of a simple emulsion.

Because of these measures, the active agent is transported throughout the entire volume of microparticles (inside the nanoparticles), which increases the amount of active agent transported.

In addition, the stability of the emulsion of the invention is particularly high, which increases the shelf life of the product between its manufacture and its use.

Also, the gaseous precursor in its liquid form acts as a barrier against the diffusion of the active agent, which avoids the involuntary release of the active agent into the patient's tissues outside of the target area irradiated with ultrasound.

Lastly, the double emulsion can carry a hydrophilic active agent as easily as a hydrophobic active agent, which means the double emulsion of the invention is highly adaptable.

In various embodiments of the emulsion of the invention, one or more of the following may be utilized:
the gaseous precursor is a fluorinated oil;
the gaseous precursor is a perfluorocarbon;
   the gaseous precursor is perfluorohexane and/or perfluoropentane;
the second emulsifier contains a fluorosurfactant;
   the fluorosurfactant contains poly (perfluoropropylene glycol) carboxylate;
   the fluorosurfactant is obtained from poly(perfluoropropylene glycol) carboxylate, perfluorocarbon, and ammonium hydroxide;
the active agent is chosen from the group consisting of markers and drugs;
the active agent is a marker chosen from the group consisting of optical dyes and contrast agents for medical imaging;
the active agent is an optical dye containing fluorescein;
the active agent is a therapeutic agent chosen from the group consisting of cancer chemotherapy agents and messenger RNA;
the inner liquid is aqueous and the active agent is hydrophilic;
the inner liquid is an oil and the active agent is hydrophobic;
the inner liquid is aqueous and the active agent is hydrophobic and encapsulated in particles smaller than 1 μm in emulsion in the inner liquid;
the diameter of the microparticles is less than 10 μm, and is advantageously approximately 5 μm; the diameter of the nanoparticles is less than 4 μm, and is advantageously approximately 0.3 to 1 μm.

Another object of the invention is a method for producing an emulsion activatable by ultrasound as defined above, comprising the following steps:

(a) preparing a primary emulsion between the inner liquid containing the active agent on the one hand, and the gaseous precursor in liquid form plus the second emulsifier on the other hand, to obtain said nanoparticles in emulsion in the gaseous precursor,

(b) preparing a secondary emulsion between the primary emulsion on the one hand and the aqueous solution plus the first emulsifier on the other hand, to obtain said microparticles in the aqueous solution.

In various embodiments of the method of the invention, one or more of the following may be utilized:
the step of preparing the secondary emulsion is done by hydrodynamic focusing in a microfluidic device at a junction between at least a first and a second microfluidic supply channels which respectively supply the primary emulsion and the aqueous solution plus the first emulsifier, said supply channels leading into a microfluidic outlet channel which carries away the emulsion activatable by ultrasound;
the first and second supply channels and the outlet channel have an inner surface which is hydrophilic at said junction;
the first supply channel and the outlet channel are each less than 20 μm wide and less than 20 μm deep at the junction between the first and second supply channels;
the first supply channel and the outlet channel are each less than 10 μm wide and less than 10 μm deep at the junction between the first and second supply channels;
the microfluidic device comprises two second supply channels which are substantially perpendicular to the first supply channel and which face each other at said junction.

### BRIEF DESCRIPTION OF THE DRAWINGS

Other features and advantages of the invention will be apparent from the following description of one of its embodiments provided as a non-limiting example, with reference to the attached drawings.

In the drawings:

FIG. 1 is a schematic view of a microparticle in emulsion in an aqueous solution, in one embodiment of the invention,

FIG. 2 is a diagram of an example of a microfluidic device for obtaining microparticles such as the one in FIG. 1 in emulsion,

FIG. 3 is a diagram showing an ultrasound device for locally activating an emulsion containing microparticles such as the one in FIG. 1, in the target areas of a patient's body,

FIG. 4 is a block diagram of the device in FIG. 3.

### DETAILED DESCRIPTION

In the different figures, the same references indicate the same or similar elements.

The invention proposes a double emulsion, which can be injected into a patient and locally activated in a target area of the patient's body by irradiating the target area with ultrasound focused on said target area.

As schematically represented in FIG. 1, this double emulsion contains a secondary emulsion of microparticles 1 in an aqueous solution 2. These microparticles 1 have a diameter D of less than 20 μm. Only one of the microparticles 1 is represented in FIG. 1 for simplicity.

The diameter D is advantageously less than 20 μm and preferably less than 10 μm, for example less than 8 μm and in particular approximately 5 μm, which allows the microparticles to circulate in the capillary vessels of a patient when the double emulsion is injected, as will be explained below.

The microparticles 1 comprise an outer wall 4 which is substantially spherical and is formed by a first emulsifier, particularly a surfactant such as “Pluronic F68®” for example.

This outer wall 4 (liquid like the wall of a bubble) encapsulates a gaseous precursor liquid 3 which is vaporizable by ultrasound (or more generally a compound activatable by ultrasound), containing a primary emulsion of nanoparticles 5. The gaseous precursor can be a fluorinated oil, particularly a perfluorocarbon such as perfluorohexane or perfluoropentane for example.

The nanoparticles 5 have a diameter of less than 5 μm and preferably 0.3-1 μm, for example about 500 nm. These nanoparticles 5 each have a substantially spherical outer wall (liquid like the wall of a bubble) which is formed by a second emulsifier, for example a fluorosurfactant such as poly(perfluoropropylene glycol) carboxylate (sold by Du Pont as “Krytox 157 FSH®”). More specifically, the fluorosurfactant can be prepared from poly(perfluoropropylene glycol) carboxylate, perfluorocarbon, and ammonium hydroxide. As an example, this surfactant can be obtained by adding 10 mg Krytox 157 FSH® and 10 ml ammonium hydroxide to 10 mg perfluorohexane (see Holze et al, “Biocompatible surfactants for water-in-fluorocarbon emulsions”, Lab Chip, 2008, 1632-1639, the Royal Society of Chemistry 2008).

The outer wall 7 encapsulates an inner liquid 6, for example water or more generally an aqueous solution, which contains an active agent, in particular a tracer or drug.

More specifically, the active agent can be:
a marker chosen from the group consisting of optical dyes (for example fluorescein) and contrast agents for medical imaging (particularly contrast agents for MRI, X-rays, ultrasounds, or other imaging);
a marker intended to act as a target for a therapeutic agent;
a therapeutic agent chosen from the group consisting of cancer chemotherapy agents, vascular targeting agents, toxins and messenger RNA, DNA, etc.

The active agent can be hydrophilic.

The active agent can be hydrophobic, in which case it can be for example:
either in solution in a non-aqueous inner liquid, for example a fluorinated oil,
or in emulsion in an aqueous inner liquid, the active agent then being encapsulated (with a fluorinated oil for example) in particles of a size less than 1 μm (for example from 0.3 to 0.4 μm) in emulsion in the inner liquid.

Considering the fact that the active agent is distributed throughout the volume of microparticles 1 (inside nanoparticles), the amount of active agent transported in the microparticles is increased in comparison to the simple emulsions activatable by ultrasound which are currently in use.

In addition, the stability of the double emulsion of the invention is particularly high because the gaseous precursor forms a barrier to the diffusion of active agents, which increases the shelf life of the product between its manufacture and its use, and avoids the involuntary release of active agent into the patient's tissues outside the target area irradiated by ultrasound.

The double emulsion described above can be obtained by a method having two basic steps:

(a) preparing a primary emulsion between the inner liquid containing the active agent on the one hand, and the gaseous precursor in liquid form plus the second emulsifier on the other hand, to obtain said nanoparticles in emulsion in the gaseous precursor,

(b) preparing a secondary emulsion between the primary emulsion on the one hand and the aqueous solution plus the first emulsifier on the other hand, to obtain said microparticles in the aqueous solution.

### Example of Preparing the Primary Emulsion

As an example, a certain initial quantity can be used of perfluorohexane or other gaseous precursor in fluorinated oil form, plus the second emulsifier, particularly a fluorosurfactant such as the one described above, obtained by adding 10 mg Krytox 157 FSH® and 10 ml ammonium hydroxide to 10 mg perfluorohexane.

One then adds to the perfluorohexane, 20% by weight of the inner liquid, for example an aqueous solution containing the active agent (for example fluorescein).

The primary emulsion is then achieved for example by shearing in a cylindrical Couette cell, for example using a Polytron PT 100® homogenizer at 15,000 rpm for 15 min.

This obtains an emulsion of nanoparticles 5 having a diameter d of about 500 nm.

This emulsion can then be centrifuged to increase the volumetric fraction of nanoparticles in the water-in-oil (up to 70% for example).

### Example of Preparing the Secondary Emulsion

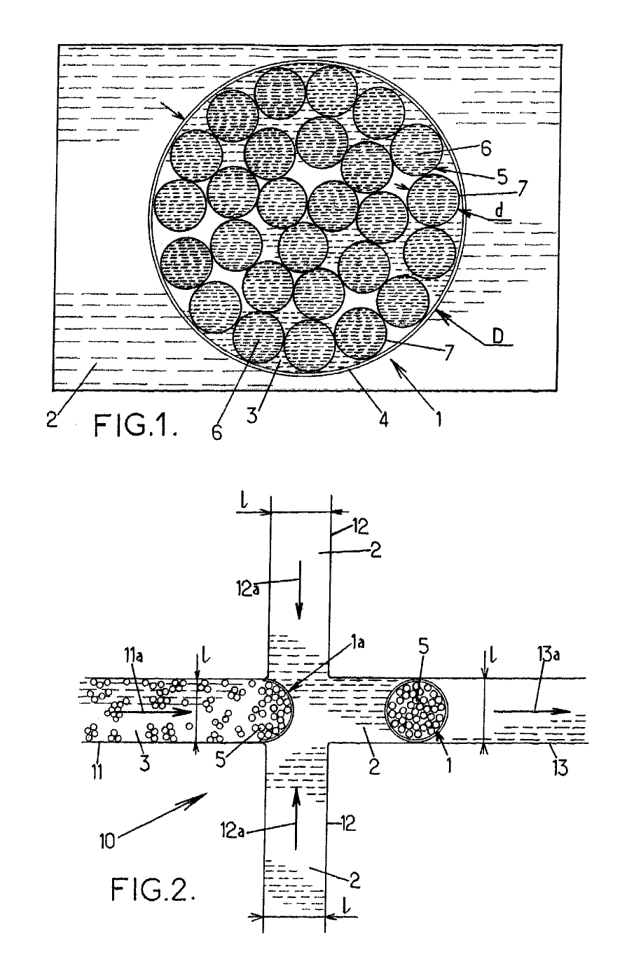

The step of preparing the secondary emulsion can be done by hydrodynamic focusing in a microfluidic device 10 such as the one represented in FIG. 2, at a junction between a first microfluidic supply channel 11 and at least one second supply channel 12 (preferably two second channels 12 facing each other and perpendicular to the first supply channel 11) which open into a microfluidic outlet channel 13 arranged for example in alignment with the first supply channel 11.

The microfluidic device can be made in particular using a soft lithographic technique with polydimethylsiloxane (PDMS), described for example by Duffy et al (“Rapid prototyping of microfluidic systems in Poly(dimethylsiloxane)” Analytical Chemistry, Vol. 70, No. 23, Dec. 1, 1998, pp 4974-4984).

The channels 11-13 can then be in the form of grooves having a rectangular cross-section, of a depth for example greater than 0.5 microns and less than 10 μm, in particular less than 10 μm or even less than 3 μm, for example approximately 2.5 μm. The depth of the channels 11-13 can advantageously be greater (for example about 30 μm) when not in proximity to the junction between the channels 11-12.

The width 1 of these channels can be less than 20 μm and in particular less than 10 μm, for example approximately 5 to 10 μm. This width 1 can be the same for all the channels 11-13 as is represented in the example, or can be different. In the latter case, the dimensions mentioned above apply at least for the channels 11, 13.

The surface treatment of the inner walls of the channels is preferably hydrophilic in order to facilitate the formation of direct emulsions.

The first supply channel 11 supplies the primary emulsion 3, 5 which flows in the direction of the arrow 11a towards the outlet channel, due to the effect of an external pressure from compressed air which can be for example approximately 5 bar.

The second supply channels 12 supply the aqueous solution 2 plus the first emulsifier (for example distilled water plus a surfactant such as Pluronic F68®, in a concentration which in particular can be 1% by weight), in the direction of the arrows 12a towards the junction with the first supply channel 11, due to the effect of external pressure from compressed air which can be for example approximately 2 bar.

The geometry of the hydrodynamic focusing forms microparticles 1 dispersed in the external aqueous phase, as represented in the diagram in FIG. 2. This geometry determines the diameter D of the microparticles 1, and the conditions of their formation ensure an excellent monodispersity of the particles 1 formed (the dispersion around D is typically less than 3%). In the present case, droplets are formed having a diameter D of about 5 μm.

The transmission frequency for the microparticles 1 in the device 10 is typically about 10 kHz.

### Application Example

When using the emulsion activatable by ultrasounds, this emulsion is injected into a patient, for example by intravenous injection, so that the microparticles 1 are diffused into all or part of the patient's body 29 by the circulatory system.

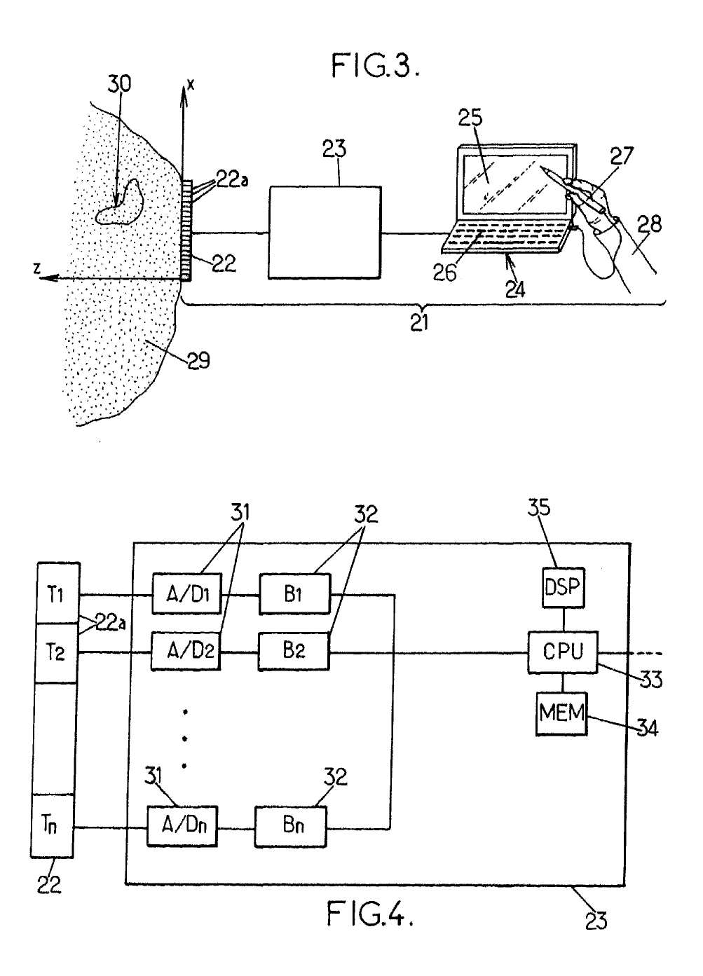

Some of these microparticles are then activated in a target area 30, for example a tumor, by causing them to rupture due to the effect of focused ultrasound emitted by an ultrasound device 21 visible in FIG. 3.

This ultrasound device 21 is an ultrasonograph comprising:
a network 22 of ultrasound transducers, for example an array of the type commonly used in ultrasonography, comprising a number n of ultrasound transducers 22a (for example about 100 to 300 transducers, transmitting at about 2.5 MHz for example).
a controller 23 which controls the network 22 of transducers during transmission and acquires the signals captured by this network,
a microcomputer 24 for controlling the controller 23, said microcomputer 24 comprising a user interface which includes a screen 25 on which ultrasound images captured by the network 22 of transducers can be viewed; said user interface also comprises a keyboard 26 for example associated with a mouse or similar device (not represented) and if applicable a pointing device 27 such as a light pen or similar device, which for example allows an operator 28 to circumscribe an area on the screen 25, as will be explained below.

The network 22 of transducers is designed to be placed in contact with a solid target medium 9, for example a portion of the body of a human or animal, in order to define and mark one or more areas of interest 30 in this medium, as will be explained below. The area of interest 30 can for example be a lesion such as a tumor.

The controller 23 and the microcomputer 24 together form a control device for controlling the network 22 of transducers and capturing and processing signals from this network. It is possible for the functions of the controller 23 and the microcomputer 24 to be carried out by a single electronic device.

As represented in FIG. 4, the controller 23 can for example comprise:
n analog-to-digital converters 31 (A/D₁-A/Dₙ) that are individually connected (for example by a cable) to the n transducers (T₁-Tₙ) of the network 22 of transducers;
n buffers 32 (B₁-Bₙ) respectively connected to the analog-to-digital converters 31,
a central processing unit 33 (CPU) communicating with the buffers 32 and the microcomputer 24,
a central memory 34 (MEM) connected to the central processing unit 33,
a digital signal processor 35 (DSP) connected to the central processing unit 33.

The device 21 can initially be used conventionally in ultrasound imaging mode, for viewing an image of the target 30 on the screen 25. The operator 2 can, for example, define the target area 30 by tracing its edges on the screen 25, for example using the abovementioned light pen 27 or any other user interface acting as a pointing device.

When the area of interest 30 has been defined by the operator, he initiates the emulsion activation step by causing the successive emission of activation ultrasound beams focused on different points of said target area 30, such that the entire target area 30 receives ultrasound which ruptures the microparticles 1 that it contains by vaporizing the fluorinated oil 3 of these microparticles. As the encapsulation of the nanoparticles 5 is then no longer effective for a gas phase, the active agent initially contained within the nanoparticles 5 is released. After this release, the active agent is dispersed into the external medium by diffusion and convection. The expansion of the vaporized phase of the microparticles 1 and the sonoporation due to the acoustic field contribute to an efficient distribution of the active agent into the tissues. When the active agent is an optical dye such as fluorescein, the tissues of the target area 30 are dyed in a lasting manner, and are therefore easily spotted by a surgeon during resection.

The pressure and duration of each activating ultrasound beam are appropriate for activating the marker without damaging the tissues of the patient 29. For example, each activating ultrasound beam has a duration of 1 to 1000 μs, in particular from 10 to 1000 μs (microseconds), and said activating ultrasound beam exerts a pressure on the tissues of less than 8 MPa, in particular less than 6 MPa (megapascals), which corresponds to conventional imaging pressures.

1. An emulsion activatable by ultrasound, comprising, in emulsion in an aqueous solution, microparticles having a diameter of less than 20 μm comprising an active agent and a gaseous precursor in a liquid form activatable by ultrasound, encapsulated by a first emulsifier,
wherein the microparticles contain nanoparticles having a diameter of less than 5 μm in emulsion in the gaseous precursor, each nanoparticle comprising an inner liquid which contains the active agent and which is encapsulated by a second emulsifier, said gaseous precursor forming a barrier against the diffusion of the active agent.

2. The emulsion according to claim 1, wherein the gaseous precursor is a fluorinated oil.

3. The emulsion according to claim 2, wherein the gaseous precursor is a perfluorocarbon.

4. The emulsion according to claim 3, wherein the gaseous precursor is perfluorohexane and/or perfluoropentane.

5. The emulsion according to claim 1, wherein the second emulsifier contains a fluorosurfactant.

6. The emulsion according to claim 5, wherein the fluorosurfactant contains poly(perfluoropropylene glycol)carboxylate.

7. The emulsion according to claim 6, wherein the fluorosurfactant is obtained from poly(perfluoropropylene glycol)carboxylate, perfluorocarbon, and ammonium hydroxide.

8. The emulsion according to claim 1, wherein the active agent is chosen from the group consisting of markers and drugs.

9. The emulsion according to claim 8, wherein the active agent is a marker chosen from the group consisting of optical dyes and contrast agents for medical imaging.

10. The emulsion according to claim 9, wherein the active agent is an optical dye containing fluorescein.

11. The emulsion according to claim 8, wherein the active agent is a therapeutic agent chosen from the group consisting of cancer chemotherapy agents and messenger RNA.

12. The emulsion according to claim 1, wherein the inner liquid is aqueous and the active agent is hydrophilic.

13. The emulsion according to claim 1, wherein the inner liquid is an oil and the active agent is hydrophobic.

14. The emulsion according to claim 1, wherein the inner liquid is aqueous and the active agent is hydrophobic and encapsulated in particles smaller than 1 μm in emulsion in the inner liquid.

15. The emulsion according to claim 1, wherein the diameter of the microparticles is less than 10 μm, advantageously approximately 5 μm, and the diameter of the nanoparticles is less than 4 μm, advantageously approximately 0.3 to 1 μm.

16. A method for producing an emulsion activatable by ultrasound according to any one of the above claims, comprising the following steps:
(a) preparing a primary emulsion between the inner liquid containing the active agent on the one hand, and the gaseous precursor in liquid form plus the second emulsifier on the other hand, to obtain said nanoparticles in emulsion in the gaseous precursor,
(b) preparing a secondary emulsion between the primary emulsion on the one hand and the aqueous solution plus the first emulsifier on the other hand, to obtain said microparticles in the aqueous solution.

17. The method according to claim 16, wherein the step of preparing the secondary emulsion is done by hydrodynamic focusing in a microfluidic device at a junction between at least a first and a second microfluidic supply channels which respectively supply the primary emulsion and the aqueous solution plus the first emulsifier, said supply channels leading into a microfluidic outlet channel which carries away the emulsion activatable by ultrasound.

18. The method according to claim 17, wherein the first and second supply channels and the outlet channel have a hydrophilic inner surface at said junction.

19. The method according to claim 17, wherein the first supply channel and the outlet channel are each less than 20 μm in width and less than 20 μm in depth at the junction between the first and second supply channels.

20. The method according to claim 19, wherein the first supply channel and the outlet channel are each less than 10 μm in width and less than 10 μm in depth at the junction between the first and second supply channels.

21. The method according to claim 17, wherein the microfluidic device comprises two second supply channels which are substantially perpendicular to the first supply channel and which face each other at said junction.
